(19) United States
(12) Patent Application Publication
(10) Pub. No.: US 2005/0130229 A1
(43) Pub. Date: Jun. 16, 2005

(54) Indexing scheme for formulation workflows

(75) Inventor: David R. Dorsett JR., Slidell, LA (US)

Correspondence Address: SYMYX TECHNOLOGIES INC;LEGAL DEPARTMENT, 3100 CENTRAL EXPRESS, SANTA CLARA, CA, 95051, US

(73) Assignee: Symyx Technologies, Inc.

(21) Appl. No.: 11/016,145

(22) Filed: Dec. 16, 2004

Related U.S. Application Data
(60) Provisional application No. 60/530,145, filed on Dec. 16, 2003.

(51) Int. Cl. C12Q 1/68; G01N 33/53

(52) U.S. Cl. 435/7.1; 702/19

(57) ABSTRACT

Methods and computer program products for managing data associated with members of related libraries of materials that include a recipient library and first and second source libraries. The members of the recipient library comprise materials derived from the first and second source libraries. An experiment object representing an experiment performed on members of the recipient library, and having a plurality of associated elements, each representing member(s) of the recipient library, is defined. A source identifier identifying a source from which the material of the corresponding recipient library member was derived is stored in association with each of the plurality of elements.

### CROSS-REFERENCE TO RELATED APPLICATIONS

This application claims the benefit of U.S. Provisional Application No. 60/530,145, filed on Dec. 16, 2003, which is incorporated by reference herein.

### BACKGROUND

This invention relates to database systems and methods for storing and manipulating experimental data.

The discovery of new materials with novel chemical and physical properties often leads to the development of new and useful technologies. Traditionally, the discovery and development of materials has been a trial and error process carried out by scientists who generate data one experiment at a time. This process suffers from low success rates, long time lines, and high costs, particularly as the desired materials increase in complexity. As a result, the discovery of new materials depends largely on the ability to synthesize and analyze large numbers of new materials. Given approximately 100 elements in the periodic table that can be used to make compositions consisting of two or more elements, an incredibly large number of possible new compounds remain largely unexplored, especially when processing variables are considered. One approach to the preparation and analysis of such large numbers of compounds has been the application of combinatorial chemistry.

In general, combinatorial chemistry refers to the approach of creating vast numbers of compounds by reacting a set of starting chemicals in many combinations. Since its introduction into the pharmaceutical industry in the late 1980s, combinatorial chemistry has dramatically sped up the drug discovery process and is now becoming a standard practice in that industry (Chem. Eng. News Feb. 12, 1996). More recently, combinatorial techniques have been successfully applied to the synthesis of inorganic materials (G. Briceno et al., SCIENCE 270, 273-275, 1995 and X. D. Xiang et al., SCIENCE 268, 1738-1740, 1995). By use of various deposition techniques, masking strategies, reaction and processing conditions, it is now possible to generate hundreds to thousands of materials of distinct compositions . These materials include biomaterials, organics, inorganics, organometallics, and polymers. Deposition techniques include a variety of thin-film deposition approaches (e.g., sputtering, ablation, evaporation) and liquid-dispensing or solid-dispensing systems as disclosed in U.S. Pat. No. 6,004,617, which is incorporated by reference herein. See also, for example, U.S. Pat. No. 5,985,356 (inorganic materials), U.S. Pat. No. 6,420,179 (organometallic materials), U.S. Pat. No. 6,346,290 (initiated polymerization), U.S. Pat. No. 6,030,917 (metal-ligand catalysts, e.g. for olefin polymerization).

The generation of large numbers of new materials presents a significant challenge for conventional analytical techniques. By applying parallel or rapid serial screening techniques to these libraries of materials, however, combinatorial chemistry accelerates the speed of research, facilitates breakthroughs, and expands the amount of information available to researchers. Furthermore, the ability to observe the relationships between hundreds or thousands of materials in a short period of time enables scientists to make well-informed decisions in the discovery process and to find unexpected trends. High throughput screening techniques have been developed to facilitate this discovery process, as disclosed, for example, in U.S. Pat. Nos. 5,959,297; 6,034,775, 6,572,750, 6,514,764, 6,187,164, 6,577,392, 6,406,632, 6,410,331, 6,149,846, 6,461,515, 6,535,284, 6,455,316, and 6,438,497, each of which is incorporated by reference herein.

The vast quantities of data generated through the application of combinatorial and/or high throughput screening techniques can overwhelm conventional data acquisition, processing, and management systems. Existing laboratory data management systems such as various Laboratory Information Management Systems (LIMS) typically provide for data acquisition, connecting analytical instruments in the lab to one or more workstations or personal computers where the data can be archived. Such systems are ill-equipped to rapidly retrieve and process the large amounts of data generated in complex workflows, such as when multiple experiments are performed on related combinatorial libraries. For data generated in a large or complex workflow, a dynamic mapping table can be used to retrieve data from a database by translating a request for data for a material in one library to a request for data for the same material in another library. However, this dynamic linkage system can be very complex and costly, especially if there are multiple or mixed levels of derivation. Data models can be tailored to fit the data resulting from different workflows. This approach can be inefficient and rigid, requiring a large number of different types of tables for analogous data. These methods impose significant limitations on throughput, both experimental and data processing, which stand in the way of the promised benefits of combinatorial techniques.

### SUMMARY

The invention provides methods, systems, and apparatus, including computer program products, for associating or representing data from experiments on related combinatorial libraries.

In general, in one aspect, the invention provides methods and apparatus, including computer program products, implementing techniques for managing data associated with members of related libraries of materials, including a recipient library, a first source library, and a second source library. The members of the recipient library comprise one or more materials derived from one or more members of the first source library and one or more materials derived from one or more members of the second source library. An experiment object that represents an experiment performed on members of the recipient library of materials is defined. The experiment object has a plurality of associated elements, and each of the plurality of elements represents one or more members of the recipient library. At least one source identifier is stored in association with each of the plurality of elements. The source identifier is associated with a given element identifying a source from which the material of the corresponding recipient library member was derived. A first source identifier identifies a member in the first source library and a second source identifier identifies a member in the second source library.

Advantageous implementations can include one or more of the following features. The recipient library can be a daughter library derived from at least one of the first and second source libraries in a daughtering operation. At least one of the first and second source libraries can be related to the recipient library by at least two degrees of relationship. At least one of the first and second source libraries can be related to the recipient library by at least three degrees of relationship. The first source library, the second source library and the recipient library can be related libraries in a defined workflow having N degrees of relationship between an original source library and the most distantly related recipient library for the defined workflow, where N is at least three or at least five.

Storing a source identifier can include determining the member in the first or second source library from which the material of the member of the recipient library corresponding to the element was derived by querying a library map object based on a recipient library identifier and a recipient library element identifier identifying the element in the recipient library, identifying the recipient library and the recipient library element identifier in the library map object, and receiving a source library identifier and a source library element identifier for the element in response to the query. The recipient library element identifier can identify a position of the corresponding member in the recipient library and the source library element identifier can identify a position in the source library from which the material of the corresponding member was derived. The library map object can include a plurality of library map elements, each library map element mapping from an element of the recipient library to an element of a source library from which the material of the corresponding recipient library member was derived.

The methods and apparatus can include receiving a request for experimental data associated with an element of a source library, querying a database of experiments based on the source library identifier of the source library and the source library element identifier of the element; and retrieving one or more data values corresponding to recipient library elements satisfying the query.

In general, in another aspect, the invention provides methods and apparatus, including computer program products, implementing techniques for managing experiment data associated with one or more recipient libraries of materials. Each library includes two or more members that comprise materials derived directly or indirectly from two or more source libraries. A request for experimental data associated with a member of a source library represented by an object in a database of experiment objects is received. Each experiment object represents an experiment involving a library of materials, and has one or more associated elements that represent members of the corresponding library. The source library is indicated by a source library identifier and a member of the source library is indicated by a source identifier. The database of experiment objects is searched based on a search query derived from the request and using the source library identifier and the source identifier. One or more elements from one or more experiment objects that represent experiments involving the recipient libraries are returned. The returned elements have element identifiers satisfying the search query.

In general, in another aspect, the invention provides methods and apparatus, including computer program products, implementing techniques for managing experiment data associated with one or more families of related libraries of materials, each family including three or more related libraries of materials. The three or more related libraries include a recipient library and two or more source libraries. Each library includes one or more members, and at least one member of the recipient library comprises materials derived directly or indirectly from members of the two or more source libraries. Data specifying a first recipient library is received. The first recipient library has members derived directly or indirectly from materials in at least a first source library and a second source library in a first family of related libraries of materials. The family of related libraries has a first library family structure defined by the relationships of at least the first recipient library, the first source library and the second source library. A plurality of elements of a first library map is defined. The plurality of elements includes a library map element identifying each member of the first recipient library. Each library map element of the first library map also identifies a member of a source library in the first library family structure from which a material was transferred to the corresponding recipient library member in one or more daughtering operations. A first experiment object is generated according to a data model representing an experiment on members of the first recipient library. The experiment object has a plurality of associated elements representing members of the first recipient library. An element identifier is assigned to each experiment element based on the source library member identified in the library map element for the recipient library member.

Advantageous implementations can include one or more of the following features. The first recipient library can be a daughter library derived from at least one of the first and second source libraries in a daughtering operation. Within the first family, at least one of the first and second source libraries can be related to the first recipient library by at least three degrees of relationship. The first source library, the second source library and the first recipient library can be related libraries in a workflow comprising N degrees of relationship between an original source library and the farthest related recipient library for the defined workflow, where N is at least three or at least five. At least one of the first and second source libraries can be related to the first recipient library by at least n degrees of relationship, where n ranges from 1 to N.

The methods and apparatus can include receiving data specifying a second recipient library. The second recipient library has members derived from materials in two or more source libraries in a second family of library family structure defined by the relationships of the three or more related libraries in the second family. The second library family structure is different than the first library family structure. A plurality of elements of a second library map are defined. The plurality of elements include a library map element identifying each member of the second recipient library. Each library map element of the second library map also identifies a member of a source library in the second library family structure from which a material was transferred to the corresponding recipient library member in one or more daughtering operations. A second experiment object is generated according to the data model representing an experiment on the second recipient library. The second experiment object has a plurality of associated elements representing members of the second recipient library. An element identifier is assigned to each experiment element of the second experiment object based on the source library member identified in the library map element for the recipient library member. One or more experimental data values can be associated with one or more elements of the experiment object. Each experimental data value represents an observation associated with the corresponding member of the first recipient library.

In general, in another aspect, the invention provides a data structure tangibly embodied in an information carrier for managing data from experiments performed on members of related libraries of materials including a recipient library and a source library. The members of the recipient library comprise one or more materials derived at least in part from members of the source library. The data structure includes an identifier for each of a plurality of members of the recipient library. A source identifier is associated with each identifier. Each source identifier identifies a source from which a material associated with the corresponding recipient library member was derived.

The invention can be implemented to realize one or more of the following advantages, alone or in the various possible combinations. The invention provides general models for associating data for materials in derivative workflows. Data from different experiments performed on a particular material can be associated with a library member from which the material was derived (e.g., even if such experiments are performed at a different time and/or different location and/or by different entities). Data for a material in a given set of libraries and experiments can be associated when libraries are created by daughtering operations. Data can be associated automatically. Data can be associated in response to a request, for example, a request for experimental data associated with a material in a library. A mapping table can be used to translate requests for data for a material in one library to requests for data for the same material in a related library. Data for a material from different experiments and libraries can be presented in a format that makes it easy to compare data from different experiments and libraries. The invention can apply to workflows that contain multiple daughter libraries having members derived from a single parent library and/or that contain individual daughter libraries having members derived from multiple parent libraries. The invention can apply to workflows that contain a sequence of daughtering operations in which at least one member of one daughter library is used as a source in a subsequent daughtering operation. The invention applies to workflows that contain an indefinite number of experiments. The invention is extensible to new classes of experiments. Although described in connection with high throughput workflows (e.g. as used in combinatorial materials science involving automated, highly-parallel synthesis and/or screening of materials) and having substantial benefit therein, the present invention is also applicable to workflows that are only partially high-throughput (e.g. automated synthesis with conventional screening) or workflows that are completely conventional.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a block diagram illustrating a laboratory data management system including a database server process according to one aspect of the invention.

FIG. 2A illustrates the creation of daughter libraries in daughtering operations in which materials in a daughter library are derived from a single source library. Materials in the source library can be created from stock materials.

FIG. 2B illustrates the creation of a first daughter library in a daughtering operation in which materials in the first daughter library are derived from two source libraries and a stock material, and materials in the source libraries are created from stock materials. A second daughter library is also created in a daughtering operation using the first daughter library as a source library.

FIG. 2C illustrates the creation of a first daughter library in a daughtering operation in which materials in the first daughter library are derived from multiple source libraries. A second daughter library is created in a daughtering operation that uses the first daughter library as a source library and locates the materials in the second daughter library differently than in the first daughter library.

FIG. 3A illustrates a simple derivative workflow where materials in each of several new libraries are derived from a single “master synthesis” source library to produce a two-level family of related libraries.

FIG. 3B illustrates a complex derivative workflow where materials in each of two new libraries are derived from two or more “master synthesis” source libraries to produce a two-level family of related libraries.

FIG. 3C illustrates a highly complex workflow where materials in each of several libraries are derived from one or two “master synthesis” source libraries; from one, two or three daughter libraries; or from a “master synthesis” source library and a daughter library to produce a four-level family of related libraries.

FIG. 4 illustrates the association of experiments and data sets with two related libraries.

FIG. 5 is a diagram of a model of experiment objects having associated experiment element objects for related libraries.

FIG. 6 is a flow chart illustrating a method using a LibraryMap Object to reference experimental data for a material in multiple related libraries.

Like reference symbols in the various drawings indicate like elements.

### DETAILED DESCRIPTION

The invention provides systems and methods for managing data from a workflow where the data are associated with members of related libraries of materials. Related libraries include materials that have been at least partially and either directly or indirectly derived from a common source library. A workflow is the set of relationships between all the activities in a research project, and defines the relationships between libraries and data created as part of that workflow.

Related libraries are produced by daughtering operations, in which at least some materials of a recipient (e.g. “daughter”) library are derived or obtained from one or more materials of one or more source libraries (e.g. “parent” libraries or higher level source libraries). Libraries in a family of related libraries can be related by varying degrees, the number of degrees ranging from a 1^{st} degree relationship between a parent library and its daughter library to an Nth degree relationship between a first or original source library created in a workflow and a recipient library derived by a longest series of N daughtering operations in the workflow involving one or more materials at least partially derived from a material of that original source library. Hence, N is an integer representing the number of degrees of relationship (i.e. the number of daughtering operation) between an original source library and a most distantly related recipient library for a given user-defined workflow. Any two libraries within the predefined workflow are related by “n” degrees, where “n” is a number between 0 (for sibling libraries derived from a common parent library in a single daughtering operation) and N for that workflow. Any particular library (or material in a particular library) can be present in more than one defined workflow. A member of a particular recipient library can include a material derived from a member of a first source library, while another member of the recipient library can include a material derived from a member of a second source library, which may or may not be related to the first.

The value of N is not narrowly critical to the invention. N is at least 1, and preferably at least 2. In some embodiments, N can be at least 3, at least 4, at least 5, at least 6, at least 7, at least 8, at least 9 or at least 10. In some embodiments, N can be even greater, including for example, an integer not less than 15, not less than 20, not less than 25, not less than 30, not less than 35, not less than 40, not less than 45 or not less than 50. In other embodiments, N can be not less than 60, not less than 70, not less than 80, not less than 90 or not less than 100. For any of these aforementioned embodiments, the maximum value of N is not limited. For example, the maximum value of N can be not more than about 1,000,000, not more than about 100,000, not more than about 10,000, not more than about 1000, not more than about 500 or not more than about 200. Hence, N can preferably range generally from 2 to about 1,000,000, from 2 to about 100,000, from 2 to about 10,000, from 2 to about 1000, from 2 to about 500 or from 2 to about 200. In particularly preferred embodiments, N can range from 2 to about 100, from 2 to about 50, from 2 to about 20 or from 2 to about 10. In other preferred embodiments, N can range from 3 to about 100, from 3 to about 50, from 3 to about 20 or from 3 to about 10.

As noted above, the number of degrees of relationship between any two libraries of the defined workflow, n, can range from 0 to N for that workflow. Hence, in some embodiments, n is at least 1, and preferably at least 2. In some embodiments, n can be at least 3, at least 4, at least 5, at least 6, at least 7, at least 8, at least 9 or at least 10. In some embodiments, n can be even greater, including for example, an integer not less than 15, not less than 20, not less than 25, not less than 30, not less than 35, not less than 40, not less than 45 or not less than 50. In other embodiments, n can be not less than 60, not less than 70, not less than 80, not less than 90 or not less than 100. For any of these aforementioned embodiments, the maximum value of n limited only by N. Hence, for example, the maximum value of n can be not more than about 1,000,000, not more than about 100,000, not more than about 10,000, not more than about 1000, not more than about 500 or not more than about 200. Therefore, n can preferably range generally from 2 to about 1,000,000, from 2 to about 100,000, from 2 to about 10,000, from 2 to about 1000, from 2 to about 500 or from 2 to about 200. In particularly preferred embodiments, n can range from 2 to about 100, from 2 to about 50, from 2 to about 20 or from 2 to about 10. In other preferred embodiments, n can range from 3 to about 100, from 3 to about 50, from 3 to about 20 or from 3 to about 10.

The correspondence of materials in the related libraries can be ascertained by storing in association with each library member (e.g., in association with a data object representing the library member) a value that indicates a source of the corresponding material (a source identifier), for example, the particular library and position in that library from which the material was derived. By using the source identifiers, data from various related libraries and experiments on those libraries can be associated for a particular material.

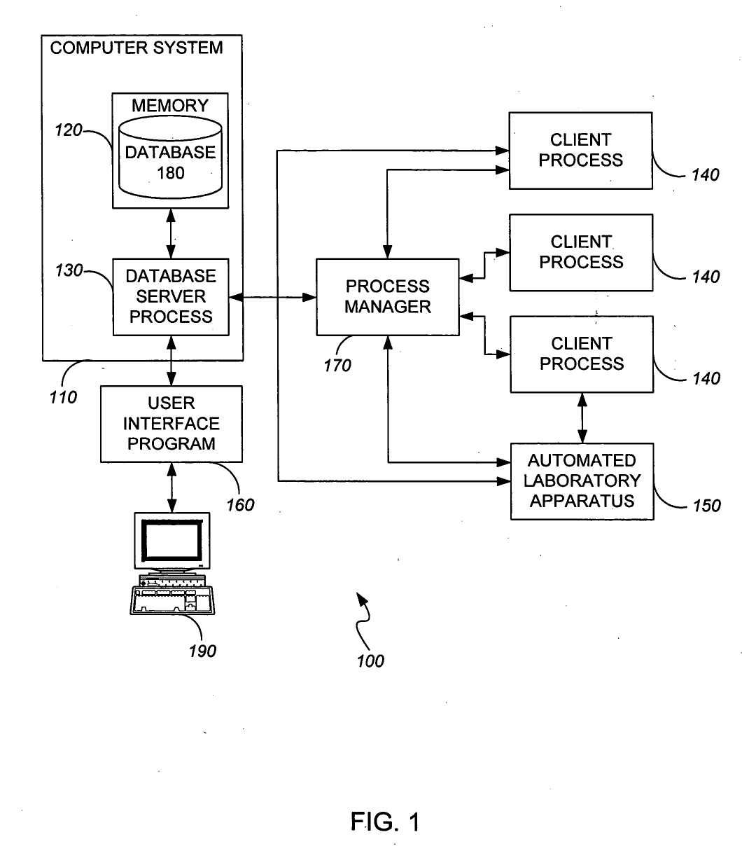

FIG. 1 illustrates a data management system 100 that includes a general-purpose programmable digital computer system 110 of conventional construction including a memory 120 and a processor for running a database server process 130, and one or more client processes 140. As used in this specification, a client process is a process that uses services provided by another process, while a server process is a process that provides such services to clients. Client processes 140 can be implemented using conventional software development tools such as Microsoft® Visual Basic®, C++, and Java™, and laboratory data management system 100 is compatible with clients developed using such tools. In one implementation, database server process 130 and client processes 140 are implemented as modules of a process control and data management program such as that described in WO 01/79949, which is incorporated by reference herein. Optionally, client processes 140 include one or more of automated or semi-automated laboratory apparatuses 150, a user interface program 160 and/or a process manager 170 for controlling laboratory apparatus 150. Exemplary laboratory apparatuses, user interface programs and process managers are described in more detail in U.S. Pat. No. 6,489,168, and WO 01/79949, each of which are incorporated by reference herein.

Laboratory data management system 100 is configured to manage data generated during the course of experiments. Database server process 130 is coupled to a database 180 stored in memory 120. In general, laboratory data management system 100 receives data from client 140 for storage, returns an identifier for the data, provides a way of retrieving the data based on the identifier, provides the ability to search the data based on the internal attribute values of the data, and provides the ability to retrieve data from these queries in a number of different ways, generally in tabular (e.g., in a relational view) and object forms. In one implementation, laboratory data management system 100 maintains three representations of each item of data: an object representation, a self-describing persistent representation, and a representation based on relational tables. Laboratory data management system 100 can be implemented as a laboratory information system as described in U.S. Pat. No. 6,658,429, which is incorporated by reference herein.

Experiments are performed, for example, by laboratory apparatus 150, on a single material or, more typically, on a set of materials such as a library of materials. A library of materials is a collection of members, typically two or more members, generally containing some variance in material composition, amount, reaction conditions, and/or processing conditions. A member typically comprises a material, where a material can be, for example, an element, chemical composition, biological molecule, or any of a variety of chemical or biological components. A combinatorial library is a set of materials prepared from chemical or biological building blocks using a combinatorial process. The library can be spatially determinant, for example, a matrix where each member represents a single constituent, location, or position on a substrate. The library can be spatially indeterminant, for example, a mixture of compounds. The library can be a conceptual collection, where each member represents, for example, data or analyses resulting from the analysis of experiments performed on samples that are not located on a common substrate, or from simulations or modeling calculations performed on hypothetical samples.

Related libraries, including source libraries and recipient libraries, can be spatially determinant, spatially indeterminant, or conceptual in nature. Members of related libraries are identifiable, e.g. capable of isolation or deconvolution, such that some or all of a material constituting a member of a source library can be transferred in one or more daughtering operations to one or more recipient libraries.

Experiments can involve the measurement of numerous variables or properties by the laboratory apparatus, as well as processing (or reprocessing) data gathered in previous experiments or otherwise obtained, such as by simulation or modeling. Typical laboratory apparatus and experimental data suitable for use in and/or manipulation by the laboratory data management systems described herein are discussed in more detail in U.S. Pat. No. 6,658,429, and U.S. application Ser. No. 09/840,003, filed Apr. 19, 2001. For example, the synthesis, characterization, and screening (i.e. testing) of materials in a combinatorial library can each constitute a separate experiment. In a synthesis experiment, materials of a library can be created, for example, by combining or manipulating chemical building blocks. In a characterization experiment, materials of the library can be observed or monitored following their creation, or features of the materials can be determined for example by calculation. In a screening experiment, materials of the library can be tested, for example, by exposure to other chemicals or conditions, and observed or monitored thereafter.

An experiment on a library is typically represented by one or more data values for one or more materials of the library. The data values representing an experiment can specify aspects of the experimental design, the methodology of the experiment, or the experimental results. The data values can, for example, name the chemicals used to create a material, specify the conditions to which the material was exposed, or describe the observable features of a material during or after its creation or manipulation. Data for a synthesis experiment can include information such as the identity, quantity, or characteristics of the chemical building blocks. Data for a characterization experiment can include a description of one of more observed properties or measured values. Data for a screening experiment can include information such as a measured concentration of solid or other constituent.

Database 180 stores experimental data, including observations, measurements, calculations, and analyses of data from experiments performed by laboratory data management system 100. The data can be of many possible data types, such as a number, a phrase, a data set, or an image. The data can be quantitative, qualitative, or Boolean. The data can be observed, measured, calculated, or otherwise determined for the experiment. The data can be for the entire library or for individual members of a library. The data can include multiple measurements for any given element or elements, as when measurements are repeated or when multiple measurements are made, for example, at different set points, different locations within a given element or elements, or at different times during the experiment.

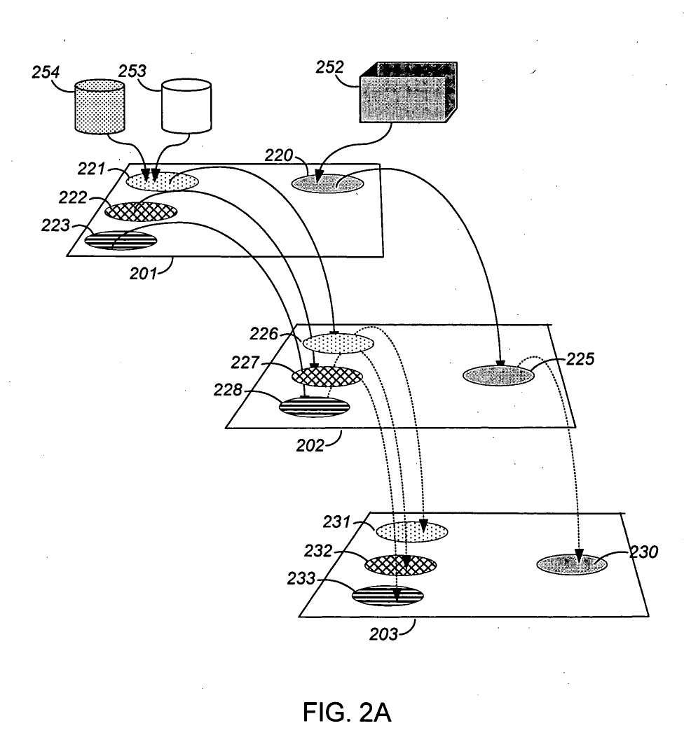

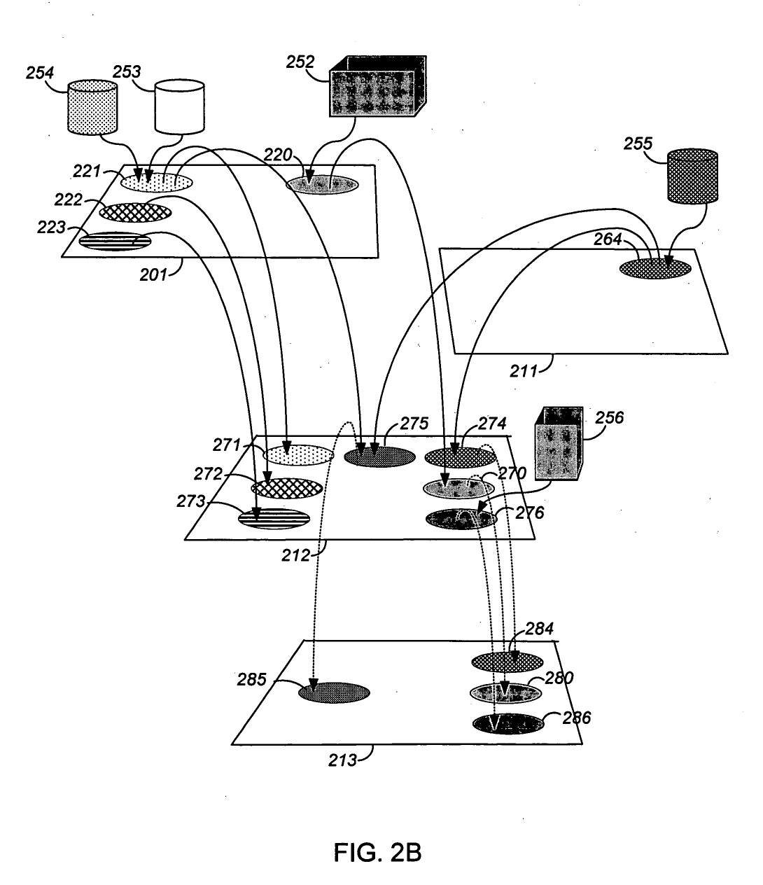

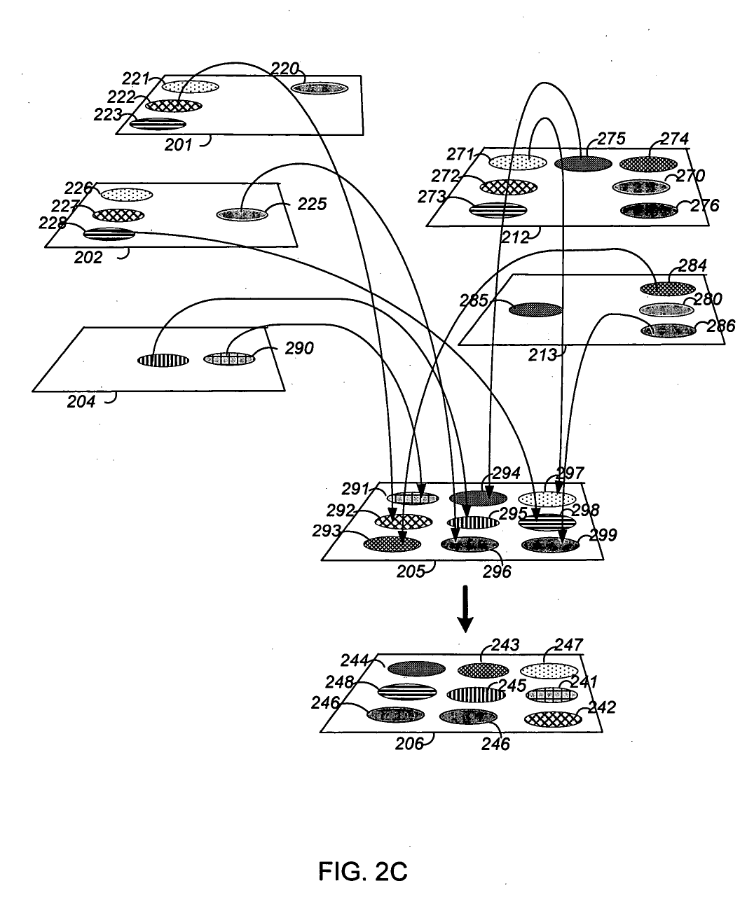

As shown in FIG. 2A, a recipient or “daughter” library 202 can be created in a daughtering operation from one or more materials in an existing library 201. A second recipient library 203 can be created in another daughtering operation using one or more materials in the first daughter library 202. The existing library 201 is a parent library with respect to the first recipient library 202; the first recipient library 202, is in turn a parent library with respect to the second recipient library 203. Thus, the second recipient library 203 is a “granddaughter” of the existing library 201. The existing library 201 is a source library with respect to both recipient libraries 202, 203 because the existing library 201 is a source of at least some of the materials for each of them. The existing library 201 can be considered a direct source of materials for the first recipient library 202, as the transfer occurred in a daughtering operation, and an indirect source of materials for the second recipient library 203, as the transfer occurred in a sequence having more than one daughtering operation.

A source library can include materials that are not associated with a related library. For example, a source library 201 can have a member 220 consisting of a material transferred from a stock material 252. Also for example, the source library can have a member 221 created by combining materials, for example, from two or more stock solutions 253, 254. A source library also can include materials that are associated with a related library. The source library 201 can have a member 222, 223 that includes a material or materials derived, as discussed in more detail below, from one or more materials in one or more related libraries, which for simplicity are not shown in FIG. 2A.

In a daughtering operation, materials from one or more members 221, 222, 223, of a parent library 201 can be transferred to a member 226, 227, 228 in a daughter library 202, for example, a member in a corresponding position on a matrix or substrate. A material from a member 220 of the parent library 201 can also be transferred to a member in a non-corresponding position 225 of the daughter library 202. Each material in the daughter library can be derived from a material in a parent library, such that the materials in the daughter library are the same as the materials in the parent library. If the parent and daughter libraries are in the form of a matrix or array, the materials in the parent and daughter libraries can have the same spatial distribution or arrangement. For example, materials at positions 225-228 of parent library 202 are transferred to corresponding positions 230-234 of its daughter library 203.However, the arrangement of materials in the daughter library can be different than the arrangement of materials in the parent library when one or more materials are transferred to non-corresponding positions in the daughter library.

Multiple recipient libraries can be created, directly or indirectly, from materials in the same source library, for example, to provide libraries for subsequent characterization, screening, or synthesis experiments. In practice, the number of recipient libraries that can be created may be physically limited by the amount of materials in the source library and the amounts transferred to each daughter library. The number of libraries in a family of related libraries is not, however, limited by application of the data models described here.

As shown in FIG. 2B, a single daughter library 212 can be created in a daughtering operation from materials in two or more parent libraries 201, 211. A material from a member of a parent library 201 can be transferred to any member in the daughter library and can be transferred to multiple members. For example, a material from a member 221, 222, 223, of the parent library 201 can be transferred to a member 271, 272, 273 in the daughter library 212, for example, a member in a corresponding position (or a non-corresponding position 220, 270) on a matrix or substrate. A material from a member 264 of a parent library 211 can be transferred to a member in a corresponding position 274 and also to a member in a non-corresponding position 275 of the daughter library 212.

A material from a member of a second parent library 211 can be transferred to the daughter library 212. For example, a material 264 in the second parent library 211 can be transferred to and constitute a member 274 of the daughter library 212. A material from one member 221 of a library 201 can be transferred to a member 275 of a daughter library 212 and combined with another material, for example, a material from a member 264 of a second library 211. In this way, a material from a member of a source library can be used as a building block for a material in a daughter library.

A daughter library 212 can have one or more members 276 each consisting of a material or materials transferred from one or more stock materials 256. In a complex workflow, a daughter library includes materials that are not all derived from a single source library. For example, the materials in a daughter library in a complex workflow can be derived from two or more source libraries or from one or more source libraries and stock materials as for libraries 210, 211, and 212 in FIG. 2B. In contrast, in a simple workflow, every material in the daughter library is derived from a material in a single source parent library, as shown in FIG. 2A and for libraries 212 and 213 in FIG. 2B, where materials 270, 274-276 in parent library 212 are transferred to members 280, 284-86 in daughter library 213.

As shown in FIG. 2C, a single daughter library 205 having materials 291-299 can be created in a daughtering operation from materials in multiple source libraries 201, 202, 204, 212, 213, where the source libraries are created and related as shown in part in FIGS. 2A and 2B. In a simple daughtering operation, a second daughter library 206 having materials 241-249 can be created from the materials 291-299 in library 205. The second daughter library 206 differs from its single parent 205 in that the locations of similar materials are different; that is, a material 241 in the second daughter library 206 derived from a material 291 in the parent library 205 is in a different location or position in the two libraries.

The number of parent libraries, P, used to create a daughter library is not narrowly critical to the invention. P is at least 1, and preferably at least 2. In some embodiments, P can be at least 3, at least 4, at least 5, at least 6, at least 7, at least 8, at least 9 or at least 10. In some embodiments, P can be even greater, including for example, an integer not less than 15, not less than 20, not less than 25, not less than 30, not less than 35, not less than 40, not less than 45 or not less than 50. In other embodiments, P can be not less than 60, not less than 70, not less than 80, not less than 90 or not less than 100. For any of these aforementioned embodiments, the maximum value of N is not limited. For example, the maximum value of P can be not more than about 1000, not more than about 500 or not more than about 200. Hence, P can preferably range generally from 2 to about 1000, from 2 to about 500 or from 2 to about 200. In particularly preferred embodiments, P can range from 2 to about 100, from 2 to about 50, from 2 to about 20 or from 2 to about 10. In other preferred embodiments, P can range from 3 to about 100, from 3 to about 50, from 3 to about 20 or from 3 to about 10.

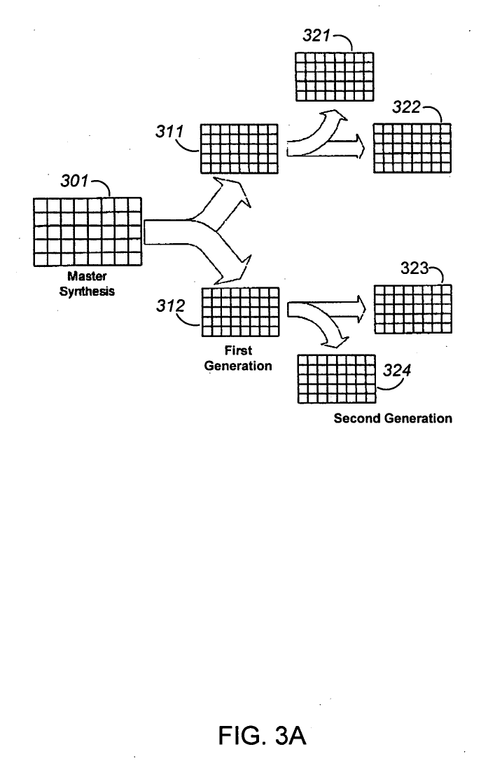

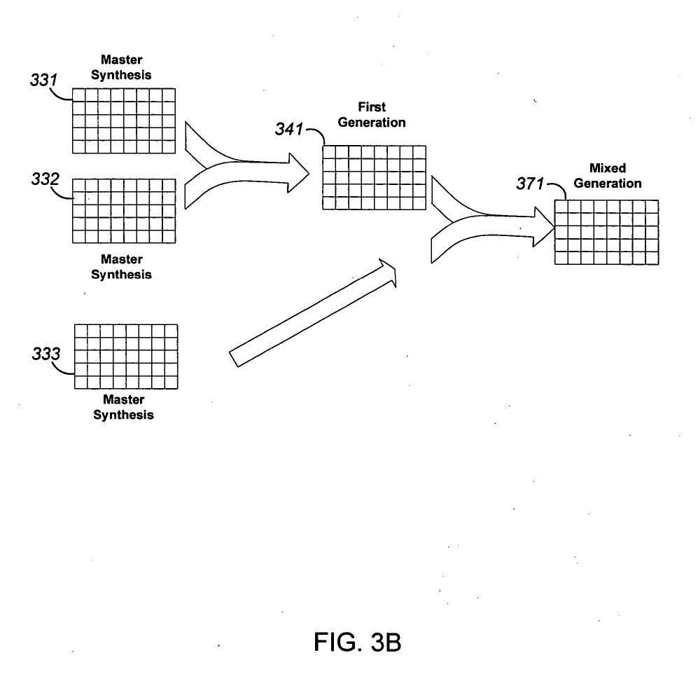

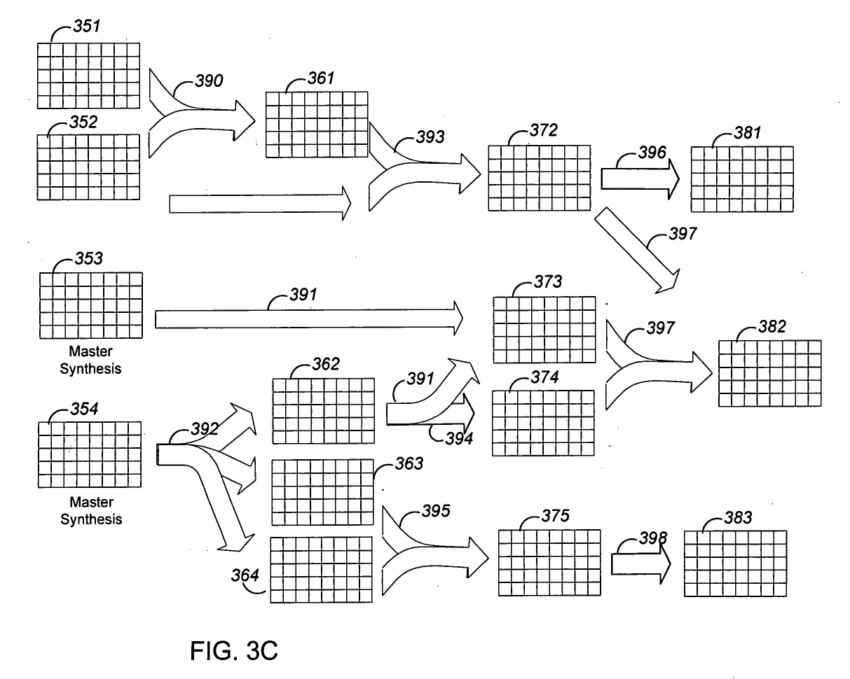

As shown in FIGS. 3A&B, a family of related libraries is characterized by a library family structure, which results from the particular workflow. A library family structure characterizes the development or creation of the family of related libraries. For example, a library family structure can trace derivations of libraries in a family of related libraries. Also for example, a library family structure can characterize, for each recipient library in the family of related libraries, the identities of its parent library or libraries. In general, a library family structure characterizes the pattern of relationships among libraries in a family of related libraries.

A simple workflow results in a library family structure where each source library is the only parent of one or more daughter libraries. In general, simple workflows result in a number of similar libraries, for which each daughter library has the same or a subset of the members of its parent. For example, as shown in FIG. 3A, two “first generation” daughter libraries 311, 312 are each created from a master synthesis library 301, for example, as discussed with respect to FIG. 2A. From each daughter library 311, 312, two additional libraries (“granddaughter” or “second generation” libraries in relation to the master synthesis library) 321-322, 323-324 are created, also for example as discussed with respect to FIG. 2A, for a total of seven related libraries.

A complex workflow results in a library family structure where each source library can be one of two or more sources (e.g. parents) of a recipient (e.g. daughter) library. In general, complex workflows result in a number of dissimilar libraries, which have various combinations of the materials present in the possible source libraries. For example, as shown in FIG. 3B, a single daughter library 341 is created from two master synthesis libraries 331, 332, for example, as described with respect to FIG. 2B. A second library 371 is created from the-daughter library 341 and a third master synthesis library 333, also for example as discussed with respect to FIG. 2B. The second library 371 is a granddaughter or second-generation library in relation to the two master synthesis libraries 331, 332, but is a daughter or first generation library in relation to the third master synthesis library 333; the second library 371 is a “mixed” generation library.

A workflow can be partially complex and partially simple, resulting in a family of libraries having complicated pattern of relationships as illustrated in FIG. 3C. A family can have any number of levels or “generations,” such as the four levels shown in FIG. 3C, wherein for example a first level includes four master synthesis libraries 351-354, a second level includes four recipient libraries 361-364, a third level includes four recipient libraries 372-375, and a fourth level includes three recipient libraries 381-383. If the degree of relationship of two libraries is determined as the number X of daughtering operations between them, and one of the two libraries is designated as level 1, then the other library is level 1+X or 1−X. For example, a sequence of three daughtering operations produces a family of libraries having four levels.

The pattern of relationships among the libraries 351-354, 361-363, 372-375, 381-383 can result, for example, from sequences of daughtering operations 390-399. The daughtering operations can include an operation 394, 396 or 398 in which materials in a library 374, 381 or 383 (respectively) are derived from materials in a single source library 362, 372 or 355 (respectively). A particular daughtering operation can be repeated. For example, a daughtering operation 392 in which materials in a library 362 are derived from materials in a single source library 354 can be repeated to create similar libraries 362, 363, 364. The daughtering operations can include an operation 390, 391, 393, 395, or 397 that combines materials from two or more libraries 351 and 352; 362 and 353; 361 and 352; 363 and 364; 372, 373 and 374 (respectively) to create recipient libraries 390, 391, 393, 395, 397 (respectively).

A family can include mixed generations, wherein a library is created from a first source library at one level in the family and a second source library at another level in the family. For example, a library 372 can be formed from materials in a first source library 361 and materials in a second source library 352, wherein materials from the first source library were derived from the materials in the second source library. Also for example, a library 373 can be formed from materials in a first source library 353 and materials in a second source library 362, wherein the first source library is a first master synthesis library and the second source library is a recipient library that was created at least in part from materials in a second master synthesis library 354.

A family can include any number of source libraries, any number of daughtering operations, and in general, any library can be a source of material, i.e. a parent, for any recipient daughter library. Accordingly, tracing the derivation of a particular material in a particular recipient library back to an early or original source library can be difficult.

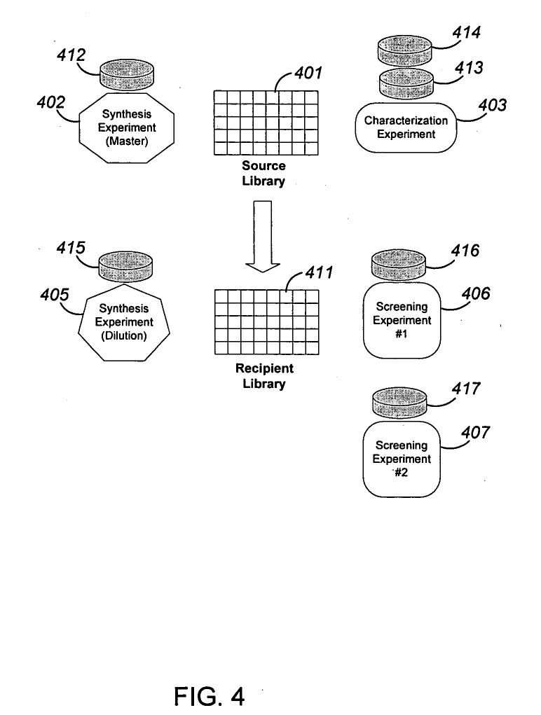

As shown in FIG. 4, multiple experiments 402-403; 405-407 can be performed on each of two related libraries 401, 411, and multiple sets of data 413, 414 can be collected for any single experiment 403. The libraries can be related simply as described with respect to FIGS. 2A & 3A or in more complex fashion as described with respect to FIGS. 2B & 3B. Materials can be synthesized in an experiment 402 on a source library 401, and one or more sets of data 412 about the synthesis can be collected. In a separate experiment 403 on the source library 401, one or more sets of data 413, 414 characterizing the materials can be collected. One or more of the materials in library 401 can be transferred to a second-generation (daughter) library 411 where. they are subject to additional experiments. For example, a set of candidate catalysts synthesized by various means can be observed and then loaded into a parallel plug-flow reactor apparatus for further testing. As shown in FIG. 4A, a set of synthesis data 415 can be collected in a synthesis experiment 405 for the daughter library 411. A first set of screening data 416 can be collected in a first screening experiment 406 on the daughter library 411, and a second set of screening data 417 can be collected in a second screening experiment 407 on the daughter library.

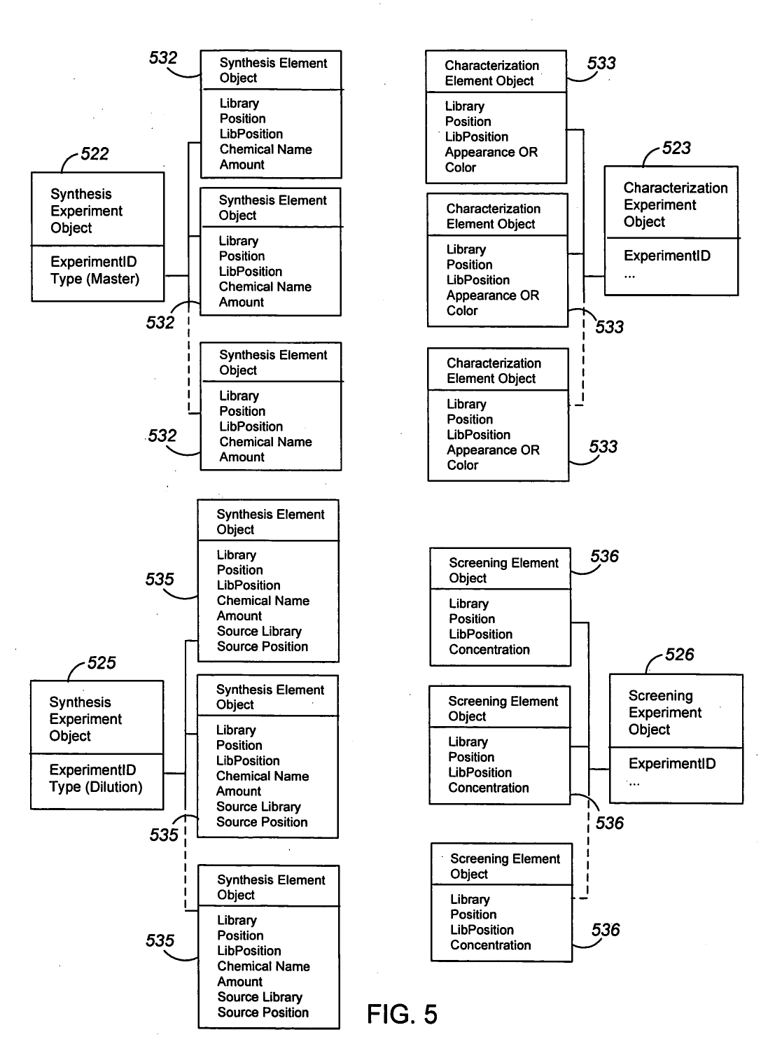

In one implementation, client processes 140 interact with experimental data generated for related libraries 201, 202; 201, 212; 301, 311; 331, 341; 401, 411 in system 100 through an object model representing experiments performed by system 100, as illustrated in FIG. 5. In this object model, an experiment performed by system 100 is represented by an experiment object 522, 523, 525, 526 having a set of associated properties and methods that represent the experiment. Each experiment object 522, 523, 525, 526 has a unique identifier or experiment ID. There are different classes of experiment object, such as Synthesis 522, 525, Characterization 523, and Screening 526. Each experiment object 522, 523, 525, 526 is associated with one or more experiment element objects 532, 533, 535, 536.The experiment element objects are typically similar across experiment classes. Typically, there is an element object for each member being studied in the experiment, although in some implementations there can be element objects for only some of the members of a library.

An experiment object can be mapped into a relational database table, for example, for ease of access or for presentation to a user. Exemplary methods for presenting data in a tabular form resembling a relational table are described in U.S. Pat. No. 6,658,429 and PCT application number WO 02/054188, which is incorporated by reference herein. Relational database tables corresponding to the experimental objects shown in FIG. 5 are discussed in more detail below.

Experimental data for materials of the source and daughter libraries that are related, for example, because a material comprising a member in the daughter library was derived in full or in part from a material comprising a member in the source library, can be associated. For example, screening data for a material in the daughter library can be associated with characterization data for the same material in the source library. In general, data for a material in one library can be associated with data for a related material in another library by using information indicative of the derivations of the materials in the libraries.

Data can be associated automatically. Data also can be associated in response to a request, such as a request for experimental data for a material in a source or daughter library. In response to such a request, the system can query a database of experiments for that member of the source or daughter library as well as related members of other libraries, and retrieve data for all such related members. An independent data structure such as the LibraryMap object discussed below can be used to identify related members of the libraries. typically, data are retrieved in system 100 from objects stored in the database 180 and presented to the requester in tabular form.

The tables below illustrate how data from experiments for specific materials in a family of related libraries can be associated according to the methods of the invention. these tables represent simplifications of the methods. Workflows and the corresponding library family structure of related libraries can be more complicated than indicated below for example, there can be several daughter libraries, and each library can be related to multiple other libraries. Data can be more substantial and extensive than shown below. For example, actual experiment data can include multiple sets of data (such as a set of spectra for each of several different wavelengths for each of the materials in a library), each of which can be stored separately, for example, in a different table. There can be many experiments performed on each library including, for example, multiple screening experiments.

An “Experiment” table provides information for each experiment performed in a work flow, information sufficient to uniquely identify the experiment and the library or libraries upon which the experiment was performed. An Experiment table can provide additional information, such as the class or type of the experiment. Each experiment is typically represented in the model by an experiment object as discussed with reference to FIG. 5. An exemplary Experiment table is illustrated in Table 1.

TABLE 1

| ID | ClassName | Type | Library |
|---|---|---|---|
| 100 | Synthesis | Master | 100000 |
| 101 | Characterization | | 100000 |
| 200 | Synthesis | Dilution | 120000 |
| 201 | Screen | | 120000 |
| 202 | Screen | | 120000 |

In the example shown in Table 1, above, the information in the Experiment table can include (1) a unique identifier for the experiment, “ID”; (2) an indicator of the class of experiment performed, “ClassName”; (3) an optional indicator of the type of experiment of a particular class, “Type”; and (3) an identifier of the library on which the experiment was preformed, “Library.” Each experiment can be represented for example in a row, and each type of information can be represented for example in a column, as shown in the table. For example, in Table 1, the experiment having ID=100 is of the class “Synthesis” and the type “Master,” and was performed on library 100000.

One or more “ExperimentClass” tables provide information for objects in each class of experiment (e.g. for each unique ClassName value) listed in the Experiment table, including for example one or more experiment objects and one or more element objects. A class of experiment can be represented in the model by several experiment and element objects corresponding, for example, to experiments performed on different libraries. There can be multiple types of experiments in a class. For example, there can a master type and a dilution type of experiment in the Synthesis class. The type of experiment in a class can be used, for example, to differentiate libraries based on their intended use.

Data from all the objects belonging to a class can be presented in a single ExperimentClass table. For example, if there are three classes of experiments in the Experiment table, there can be three ExperimentClass tables (a “SynthesisClass” table, a “CharacterizationClass” table, and a “ScreenClass” table), as shown below.

A SynthesisClass table represents information for objects in a “Synthesis” class of experiment, including information identifying the experiment and the library upon which it was performed, and data relating to the synthesis of one or more members of the library such as the identity and amount of materials used in the synthesis. An exemplary SynthesisClass table is illustrated in Table 2.

TABLE 2

| | | | | Chemical | | Source | Source |
|---|---|---|---|---|---|---|---|
| Library | Position | LibPosition | Experiment | Name | Amount | Library | Position |
| 100000 | 1 | 1000000001 | 100 | Chem A | 10 | | |
| 100000 | 1 | 1000000001 | 100 | Chem B | 10 | | |
| 100000 | 2 | 1000000002 | 100 | Chem A | 10 | | |
| 100000 | 2 | 1000000002 | 100 | Chem B | 100 | | |
| 100000 | 3 | 1000000003 | 100 | Chem A | 10 | | |
| 100000 | 3 | 1000000003 | 100 | Chem C | 10 | | |
| 100000 | 4 | 1000000004 | 100 | Chem A | 10 | | |
| 100000 | 4 | 1000000004 | 100 | Chem C | 100 | | |
| 120000 | 1 | 1200000001 | 200 | 100000-4 | 10 | 100000 | 4 |
| 120000 | 2 | 1200000002 | 200 | 100000-4 | 10 | 100000 | 4 |
| 120000 | 3 | 1200000003 | 200 | 100000-3 | 10 | 100000 | 3 |
| 120000 | 4 | 1200000004 | 200 | 100000-3 | 10 | 100000 | 3 |
| 120000 | 5 | 1200000005 | 200 | 100000-2 | 10 | 100000 | 2 |
| 120000 | 6 | 1200000006 | 200 | 100000-2 | 10 | 100000 | 2 |
| 120000 | 7 | 1200000007 | 200 | 100000-1 | 10 | 100000 | 1 |
| 120000 | 8 | 1200000008 | 200 | 100000-1 | 10 | 100000 | 1 |

In the example shown in Table 2, above, the information in the SynthesisClass table can include, for each material synthesized, (1) an identifier of the library to which the material belongs, “Library”; (2) if applicable, an identifier of the position of the material in the library, “Position”; (3) a single-column index value formed from the Library and, if applicable, Position values, “LibPosition”; (4) a unique identifier for the synthesis experiment being recorded, “ID”; (5) a descriptive name of the material used in the creation of the library element, “Chemical Name”; (6) the amount of the material used, “Amount”; (7) if applicable, the identifier of the library from which the material was derived, “Source Library”; and (8) if applicable, the identifier of the position of the material in the source library, “Source Position”. For example, as shown in the first two rows of Table 2, 10 units of Chem A and 10 units of Chem B were put in position 1 of library 100000 in synthesis experiment having ID=100.

In the SynthesisClass table, the ChemicalName can provide a source identifier. For example, if a material used to create a library member originates from a stock solution or purchase of material, its ChemicalName can be represented by a descriptive name, as described above, or by other information about the source. If a material is derived from a member of another library, for example, from a library-to-library transfer, its ChemicalName can be represented by information about the source library and position. For example, in Table 2, the last eight materials, which are all members of a daughter library (Library 120000), were derived from materials in a source library (Library 100000). The ChemicalName of each of these eight materials is replaced with a source identifier, in this case, a single-column index value formed from an identifier of the library from which the material was derived (Source Library) and the position in that library of the source material (Source Position).

A CharacterizationClass table represents information for objects in a “Characterization” class of experiment, including information identifying the experiment and the library upon which it was performed, and data characterizing one or more members of the library. One example of a CharacterizationClass table is illustrated in Table 3.

TABLE 3

| Library | Position | LibPosition | Experiment | Observation |
|---|---|---|---|---|
| 100000 | 1 | 1000000001 | 101 | Suspension |
| 100000 | 2 | 1000000002 | 101 | Clear |
| 100000 | 3 | 1000000003 | 101 | Clear |
| 100000 | 4 | 1000000004 | 101 | Clear |

In the example shown in Table 3, above, the information in the Characterization Class table can include, for each material being characterized, (1) an identifier of the library to which the material belongs, “Library”; (2) if applicable, an identifier of the position of the material in the library, “Position”; (3) a single-column index valued from the Library and, if applicable, Position values, “LibPosition”; (4) a unique identifier for the characterization experiment being recorded, “ID”; and (5) experiment values for or observations of the material. Characterization data is typically collected only for materials in parent or synthesis libraries such as library 100000. For example, in Table 3, the material at position 1 of library 100000 in experiment having ID=100 was to be in suspension.

A ScreenClass table represents information for objects in a “Screen” class of experiment, including information identifying the experiment and the library upon which it was preformed, and one or more figures of merit for one or more members of the library. An example of a ScreenClass table is illustrated in Table 4.

TABLE 4

| | | | | Figure of |
|---|---|---|---|---|
| Library | Position | LibPosition | Experiment | Merit |
| 120000 | 1 | 1200000001 | 201 | 30 |
| 120000 | 2 | 1200000002 | 201 | 32 |
| 120000 | 3 | 1200000003 | 201 | 5 |
| 120000 | 4 | 1200000004 | 201 | 4.5 |
| 120000 | 5 | 1200000005 | 201 | 55 |
| 120000 | 6 | 1200000006 | 201 | 53 |
| 120000 | 7 | 1200000007 | 201 | 6 |
| 120000 | 8 | 1200000008 | 201 | 5 |

In the example shown in Table 4, above, the information in the ScreenClass table can include, for each material being screened (1) an identifier of the library to which the material belongs, “Library”; (2) if applicable, an identifier of the position of the material in the library, “Position”; (3) a single-column index value formed from the Library and, if applicable, Position values, “LibPosition”; (4) a unique identifier for the screen experiment being recorded, “ID”; and (5) a figure of merit for the screen, such as the intensity of color of a solution. For example, as shown in Table 4, the material at position 1 of library 120000 in experiment having ID=201 had a concentration of solid in solution of 30 units.

A second set of data can be collected for an experiment. For example, a second measured feature of a screen, such as the hue or color of the solid in solution, can be recorded. As demonstrated below, data for a given experiment can be associated with other data for that experiment, for example, by (1) determining the experiment table or tables having that experiment ID(s); and (2) linking data from those tables using the LibPosition values in a relational equijoin. An exemplary table, Table 5, that associates data for experiment having ID=201 is shown below. In this table, the material at position 1 of library 120000 in experiment having ID=201 appeared yellow and had an intensity of 30 units.

TABLE 5

| Library | Position | LibPosition | Experiment | Intensity | Color |
|---|---|---|---|---|---|
| 120000 | 1 | 1200000001 | 201 | 30 | yellow |
| 120000 | 2 | 1200000002 | 201 | 32 | white |
| 120000 | 3 | 1200000003 | 201 | 5 | pink |
| 120000 | 4 | 1200000004 | 201 | 4.5 | yellow |
| 120000 | 5 | 1200000005 | 201 | 55 | yellow |
| 120000 | 6 | 1200000006 | 201 | 53 | pink |
| 120000 | 7 | 1200000007 | 201 | 6 | white |
| 120000 | 8 | 1200000008 | 201 | 5 | white |

All experiments performed on members of a library can be identified, for example, determining the set of all unique ClassName values from the Experiment table for a given library ID. The data for different experiments on a given library can be associated, for example, by (1) determining the set of library-specific tables based on the library identifier, (2) juxtaposing data from those tables using the LibPosition values.

The result of juxtaposing data from experiment tables according to the LibPosition values is shown in Table 6 below. Table 6 associates data from the synthesis and characterization experiments on library 100000, and associates data from the synthesis and screening experiments for library 120000. Relational join is not used to produce Table 6 because the number of rows for a given experiment-library-position in one table is not the same as the number of rows for that experiment-library-position in another table.

TABLE 6

| | | | Synthesis | | | | | Characterization | | Screening | |
|---|---|---|---|---|---|---|---|---|---|---|---|
| | | | Syn | Chemical | | Source | Source | Char | | Screen | |
| Library | Position | LibPosition | Exp | Name | Amount | Library | Position | Exp | Appearance | Exp | Conc |
| 100000 | 1 | 1000000001 | 100 | Chem A | 10 | | | 101 | Suspension | | |
| 100000 | 1 | 1000000001 | 100 | Chem B | 10 | | | | | | |
| 100000 | 2 | 1000000002 | 100 | Chem A | 10 | | | 101 | Clear | | |
| 100000 | 2 | 1000000002 | 100 | Chem B | 100 | | | | | | |
| 100000 | 3 | 1000000003 | 100 | Chem A | 10 | | | 101 | Clear | | |
| 100000 | 3 | 1000000003 | 100 | Chem C | 10 | | | | | | |
| 100000 | 4 | 1000000004 | 100 | Chem A | 10 | | | 101 | Clear | | |
| 100000 | 4 | 1000000004 | 100 | Chem C | 100 | | | | | | |
| 120000 | 1 | 1200000001 | 200 | 100000-4 | 10 | 100000 | 4 | | | 201 | 30 |
| 120000 | 2 | 1200000002 | 200 | 100000-4 | 10 | 100000 | 4 | | | 201 | 32 |
| 120000 | 3 | 1200000003 | 200 | 100000-4 | 10 | 100000 | 3 | | | 201 | 5 |
| 120000 | 4 | 1200000004 | 200 | 100000-3 | 10 | 100000 | 3 | | | 201 | 4.5 |
| 120000 | 5 | 1200000005 | 200 | 100000-2 | 10 | 100000 | 2 | | | 201 | 55 |
| 120000 | 6 | 1200000006 | 200 | 100000-2 | 10 | 100000 | 2 | | | 201 | 53 |
| 120000 | 7 | 1200000007 | 200 | 100000-1 | 10 | 100000 | 1 | | | 201 | 6 |
| 120000 | 8 | 1200000008 | 200 | 100000-1 | 10 | 100000 | 1 | | | 201 | 5 |

As shown in Table 6, data for experiments on library 100000 are associated by juxtaposing characterization data for a library member with one of the two lines of synthesis data for that library member. For example, there are two rows for position 1 of library 100000 in Table 2, but only one row for position 1 of library 100000 in Table 3. In the resulting table, the material at position 1 of library 100000 was synthesized in the experiment having ID=100 using 10 units of Chem A (as shown Table 2 and the first row of Table 6) and 10 units of Chem B (as shown in Table 2 and the second row of Table 6), and was characterized in experiment having ID=101 as being yellow and in suspension (as shown in Table 3 and the first row in Table 6). The information from Table 3 could be shown in the second row of Table 6.

The associations shown in the table above make it easy to see and compare values from different experiments for a material in a library. However, the usefulness of the display is limited because data from experiments on materials in Library 120000 cannot be compared easily with data from experiments on corresponding materials in Library 100000. For example, data from the screening of a material in Library 120000 is not easily compared to data from the synthesis and characterization of that material in Library 100000 because the data are far apart, in this case, in different columns and rows in the table.

Data for a particular material can be associated across experiments and libraries when libraries are created by daughtering operations. In general, to associate data from related libraries, it is necessary to “translate” member identifications for one library into member identifications for another library. For example, when the material used to create a member of a daughter library is derived solely or in part from a member of a source library, the material that constitutes the member of the daughter library can be the same as or at least correspond to the material in the source library, for example, because the material from the member of the source library is a constituent of the material in the member of the daughter library. The identifier of a member of a daughter library containing a material derived from a member of a source library can be translated into an identifier of the member of the source library from which the material was derived.

The Source Library and Source Position columns for a member of a daughter library can be used to translate the identifiers of its members into an identifier of the source library materials from which the corresponding daughter library member was derived. For example, in the table shown above, the material in library 120000 at position 8, having LibPosition 1200000008, was derived from the material at position 1 in library 100000. The records for this material—the last row in the table above—can be referred in such a way that the library and position fields, or the LibPosition field indicates the library and position of the source material rather than the library and position of the daughter of the library. In this way, the Source Library and Source Position columns provide inter-library mappings according to the derivation of the libraries during the workflow.

Using such mappings, experimental data for a material in one library can be associated with experimental data for a corresponding material in another library. For example, as shown in Table 7 below, data for materials from the synthesis and characterization experiments on a parent library can be associated with data for the corresponding materials from a screening experiment on a daughter library. In this table, data from a screening experiment on LibPosition 1200000007 and 1200000008 (as shown in the last two rows of the preceding table) is associated with data from a characterization experiment on LibPosition 1000000001 (as shown in the first row of the preceding table) by juxtaposing the data in a first entry (which in this case extends for some fields across three rows of the new table).

TABLE 7

| | | | Syn | Syn | Chemical | | Char | | Scn | |
|---|---|---|---|---|---|---|---|---|---|---|
| Lib | Position | LibPosition | Exp | Type | Name | Amt | Exp | Appearance | Exp | Conc |
| 100000 | 1 | 1000000001 | 100 | Master | Chem A | 10 | 101 | Suspension | 201 | 6 |
| | | | 100 | Master | Chem B | 10 | | | 201 | 5 |
| | | | 200 | Dilution | 100000-1 | 10 | | | | |
| 100000 | 2 | 1000000002 | 100 | Master | Chem A | 10 | 101 | Clear | 201 | 55 |
| | | | 100 | Master | Chem B | 100 | | | 201 | 53 |
| | | | 200 | Dilution | 100000-2 | 10 | | | | |
| 100000 | 3 | 1000000003 | 100 | Master | Chem A | 10 | 101 | Clear | 201 | 5 |
| | | | 100 | Master | Chem C | 10 | | | 201 | 4.5 |
| | | | 200 | Dilution | 100000-3 | 10 | | | | |
| 100000 | 4 | 1000000004 | 100 | Master | Chem A | 10 | 101 | Clear | 201 | 30 |
| | | | 100 | Master | Chem C | 100 | | | 201 | 32 |
| | | | 200 | Dilution | 100000-4 | 10 | | | | |

When a family of related libraries is characterized by multiple generations, resulting from multiple and sequential derivation, multiple translations or “links” may be used to relate the data associated with different libraries. For example, the identifier for an element corresponding to a material in a third generation library can be translated into a second identifier of the element corresponding-to the material in the second generation library from which it was derived. That second identifier can then be translated into the identifier of an element corresponding to a material in the first generation source library from which the material in the second generation library was derived. With this step-by-step approach, in a series of n libraries that are related by daughtering one from another in n−1 daughtering operations, n−1 links are needed to associate data from the source library with data for the nth recipient library.

Such links among data associated with different experiments or libraries can be provided dynamically. For example, a dynamic mapping table can be used to respond to queries and retrieve data from the database by translating a request for data for a material in one library to a request for data for the same material in another library. The queries in such a dynamic linkage system can be highly complex and costly, especially if there are multiple or mixed levels of derivation. In addition, when workflows are large or complex, data are typically highly dispersed and, it may not be desirable to follow the linkages reflecting the workflow.

Data models can be tailored to fit the data resulting from different workflows. For example, a first data model can be structured for a simple workflow involving three libraries on three levels of derivation, and a second data model can be structured for a complex workflow involving three libraries on two levels. This approach can be inefficient and rigid. For example, a given type of experiment may be performed on a library in the simple workflow and a library in the complex workflow. However, the data storage for the experiment must be implemented redundantly in each data model. As a result, there may be a large number of types of tables, and analogous data may be highly dispersed among a variety of models.

As described in more detail below, a LibraryMap object can be used to express the linkages between library members efficiently and generally, with consistency and reproducibility across data models and applications. The LibraryMap object is separate from other identifiers of a member, for example, in the synthesis table, the identifier of the member of the library from which the material was derived. The separate storage of the linkage information provides considerable flexibility. In particular, links are possible for workflows having any number of levels of derivation and any number of characterization and screening experiments. In addition, the LibraryMap object is easily extended to encompass new classes of experiments. The LibraryMap object permits association of data for selected libraries without retracing an entire lineage—that is, intervening libraries in the family of related libraries can be skipped in the association step.

The LibraryMap object is used to redefine the entries for the LibPosition index field in the tables for the daughter library. The entries are redefined to be the Library-Position associated with the source data. For example, the LibraryMap object can define the relationships between source library elements and derived library elements as follows:
SourceLibraryID←→DaughterLibraryID
SourceLibraryPosition←→DaughterLibraryPosition
   As data for a member of a daughter library arrives in the system, the LibraryMap object can be consulted. The member of the daughter library is identified, for example, by a DaughterLibraryID and DaughterLibraryPosition. If there is no entry in the LibraryMap object for the DaughterLibraryID and DaughterLibraryPosition, the LibPosition value is created from the experiment Library and the element position, as shown in the example tables above. If there is an entry for the DaughterLibraryID and DaughterLibraryPosition in the LibraryMap object, the corresponding SourceLibraryID and SourceLibraryPosition are used to determine the LibPosition value to be stored with the element data.

The tables below show a mapping table, or LibraryMap table, Table 8, for the example described in the tables above, and the SynthesisElement and ScreenElement tables, Tables 10 and 11, respectively, that result from use of the LibraryMap table. As shown in Tables 10 and 11, the LibPosition values for the elements corresponding to members of the daughter library, 120000, refer to members of the source library, 100000, from which the members of the daughter library were derived.

TABLE 8

| Source | Source | Destination | Destination |
|---|---|---|---|
| Library | Position | Library | Position |
| 100000 | 4 | 120000 | 1 |
| 100000 | 3 | 120000 | 2 |
| 100000 | 2 | 120000 | 3 |
| 100000 | 1 | 120000 | 4 |

TABLE 9

| | | | | Chemical | | Source | Source |
|---|---|---|---|---|---|---|---|
| Library | Position | LibPosition | Experiment | Name | Amount | Library | Position |
| 100000 | 1 | 1000000001 | 100 | Chem A | 10 | | |
| 100000 | 1 | 1000000001 | 100 | Chem B | 10 | | |
| 100000 | 2 | 1000000002 | 100 | Chem A | 10 | | |
| 100000 | 2 | 1000000002 | 100 | Chem B | 100 | | |
| 100000 | 3 | 1000000003 | 100 | Chem A | 10 | | |
| 100000 | 3 | 1000000003 | 100 | Chem C | 10 | | |
| 100000 | 4 | 1000000004 | 100 | Chem A | 10 | | |
| 100000 | 4 | 1000000004 | 100 | Chem C | 100 | | |
| 120000 | 1 | 1000000004 | 200 | 100000-4 | 10 | 100000 | 4 |
| 120000 | 2 | 1000000004 | 200 | 100000-4 | 10 | 100000 | 4 |
| 120000 | 3 | 1000000003 | 200 | 100000-3 | 10 | 100000 | 3 |
| 120000 | 4 | 1000000003 | 200 | 100000-3 | 10 | 100000 | 3 |
| 120000 | 5 | 1000000002 | 200 | 100000-2 | 10 | 100000 | 2 |
| 120000 | 6 | 1000000002 | 200 | 100000-2 | 10 | 100000 | 2 |
| 120000 | 7 | 1000000001 | 200 | 100000-1 | 10 | 100000 | 1 |
| 120000 | 8 | 1000000001 | 200 | 100000-1 | 10 | 100000 | 1 |

TABLE 10

| Library | Position | LibPosition | Experiment | Concentration |
|---|---|---|---|---|
| 120000 | 1 | 1000000004 | 201 | 30 |
| 120000 | 2 | 1000000004 | 201 | 32 |
| 120000 | 3 | 1000000003 | 201 | 5 |
| 120000 | 4 | 1000000003 | 201 | 4.5 |
| 120000 | 5 | 1000000002 | 201 | 55 |
| 120000 | 6 | 1000000002 | 201 | 53 |
| 120000 | 7 | 1000000001 | 201 | 6 |
| 120000 | 8 | 1000000001 | 201 | 5 |

The re-definition of the LibPosition values does not change the experiment and experiment-library links discussed above, the process of data retrieval, or the nature of the workflow on the materials. The re-definition process allows the screening data from separate experiments to be collected within what appears to be a single screening experiment. Thus, data are easily and readily compared. The re-definition process also provides flexibility in the determination of whether and where the linkages begin. For example, an initial preparatory step can be disregarded (skipped) if there are multiple steps or experiments, by defining the linkages to exclude that step. Thus, the data to be presented and compared can be selected.

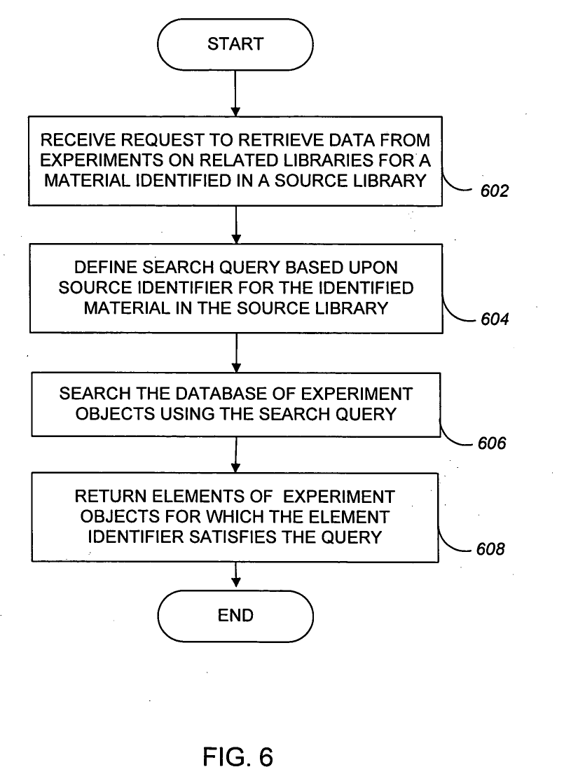

With the use of the LibraryMap object as described above, the system 100 can respond to queries for data associated with a material in a family of libraries as shown in FIG. 6. In step 602, the system receives a request to retrieve data from one or more experiments on related libraries. The request specifies a material by a source identifier. For example, the request specifies a SourceLibraryID and a SourceLibraryPosition. In step 604, the system defines a search query for the request. The search query typically requires the presence of the source identifier to be present in elements that will be returned by the search. In step 605, the system searches the database of experiment objects, including the element objects associated with the experiment objects, using the search query. For example, the system searches for all experiment elements having an identifier that is equal to or can be translated into the source identifier. In step 608, the search results are returned to the requester.

The system can also respond to requests that specify a material as a member of a daughter library, for example, by specifying an identifier of the daughter library and a position in the daughter library. The system can define a search query for a request for a material in a daughter library, for example, by identifying the source for the material and requiring the source identifier to be present in elements that will be returned by the search.

The invention and all of the functional operations described in this specification can be implemented in digital electronic circuitry, or in computer hardware, firmware, software, or in combinations of them. Apparatus of the invention can be implemented in a computer program product tangibly embodied in a machine-readable storage device for execution by a programmable processor; and method steps of the invention can be performed by a programmable processor executing a program of instructions to perform functions of the invention by operating on input data and generating output. The invention can be implemented advantageously in one or more computer programs that are executable on a programmable system including at least one programmable processor coupled to receive data and instructions from, and to transmit data and instructions to, a data storage system, at least one input device, and at least one output device. Each computer program can be implemented in a high-level procedural or object-oriented programming language, or in assembly or machine language if desired; and in any case, the language can be a compiled or interpreted language. Suitable processors include, by way of example, both general and special purpose microprocessors. Generally, a processor will receive instructions and data from a read-only memory and/or a random access memory. The essential elements of a computer are a processor for executing instructions and a memory. Generally, a computer will include one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM disks. Any of the foregoing can be supplemented by, or incorporated in, ASICs (application-specific integrated circuits).

To provide for interaction with a user, the invention can be implemented on a computer system having a display device such as a monitor or LCD screen for displaying information to the user and a keyboard and a pointing device such as a mouse or a trackball by which the user can provide input to the computer system. The computer system can be programmed to provide a graphical user interface through which computer programs interact with users.

A number of embodiments of the invention have been described. Nevertheless, it will be understood that various modifications may be made without departing from the spirit and scope of the invention. Accordingly, other embodiments are within the scope of the following claims.

1. A computer-implemented method for managing data associated with members of related libraries of materials including a recipient library, a first source library, and a second source library, the members of the recipient library comprising one or more materials derived from one or more members of the first source library and one or more materials derived from one or more members of the second source library, the method comprising:
defining an experiment object representing an experiment performed on members of the recipient library of materials, the experiment object having a plurality of associated elements, each of the plurality of elements representing one or more members of the recipient library; and
storing at least one source identifier in association with each of the plurality of elements, the source identifier associated with a given element identifying a source from which the material of the corresponding recipient library member was derived, a first source identifier identifying a member in the first source library and a second source identifier identifying a member in the second source library.

2. The method of claim 1, wherein:
the recipient library is a daughter library derived from at least one of the first and second source libraries in a daughtering operation.

3. The method of claim 1, wherein:
at least one of the first and second source libraries is related to the recipient library by at least two degrees of relationship.

4. The method of claim 1, wherein:
at least one of the first and second source libraries is related to the recipient library by at least three degrees of relationship.

5. The method of claim 1, wherein:
the first source library, the second source library and the recipient library are related libraries in a defined workflow having N degrees of relationship between an original source library and the most distantly related recipient library for the defined workflow, N being at least three.

6. The method of claim 5, wherein:
N is at least five.

7. The method of claim 1, wherein:
storing a source identifier in association with an element includes, for an element representing one of the one or more members, determining the member in the first or second source library from which the material of the member of the recipient library corresponding to the element was derived by:
querying a library map object based on a recipient library identifier and a recipient library element identifier identifying the element in the recipient library, and
receiving a source library identifier and a source library element identifier for the element in response to the query.

8. The method of claim 7, wherein:
the recipient library element identifier identifies a position of the corresponding member in the recipient library and the source library element identifier identifies a position in the source library from which the material of the corresponding member was derived.

9. The method of claim 7, wherein:
the library map object includes a plurality of library map elements, each library map element mapping from an element of the recipient library to an element of a source library from which the material of the corresponding recipient library member was derived.

10. The method of claim 1, further comprising:
receiving a request for experimental data associated with an element of the first or second source library;
querying a database of experiments based on the source library identifier of the source library and the source library element identifier of the element; and
retrieving one or more data values corresponding to recipient library elements satisfying the query.

11. A computer-implemented method for managing experiment data associated with one or more recipient libraries of materials, each library including two or more members, the recipient library members comprising materials derived directly or indirectly from two or more source libraries, the method comprising:
receiving a request for experimental data associated with a member of a source library represented by an object in a database of experiment objects, each experiment object representing an experiment involving a library of materials, each experiment object having one or more associated elements representing members of the corresponding library, the source library being indicated by a source library identifier and a member of the source library being indicated by a source identifier;
searching the database of experiment objects based on a search query derived from the request and using the source library identifier and the source identifier; and
returning one or more elements from one or more experiment objects representing experiments involving the recipient libraries, the returned elements having element identifiers satisfying the search query.

12. A computer-implemented method for managing experiment data associated with one or more families of related libraries of materials, each family including three or more related libraries of materials, the three or more related libraries including a recipient library and two or more source libraries, each library including one or more members, at least one member of the recipient library comprising materials derived directly or indirectly from members of the two or more source libraries, the method comprising:
receiving data specifying a first recipient library, the first recipient library having members derived directly or indirectly from materials in at least a first source library and a second source library in a first family of related libraries of materials, the family of related libraries having a first library family structure defined by the relationships of at least the first recipient library, the first source library and the second source library;
defining a plurality of elements of a first library map, the plurality of elements including a library map element identifying each member of the first recipient library, each library map element also identifying a member of a source library from which a material was transferred to the corresponding recipient library member in one or more daughtering operations; and
generating a first experiment object according to a data model representing an experiment on members of the first recipient library, the experiment object having a plurality of associated elements representing members of the first recipient library, the generating including assigning to each experiment element an element identifier based on the source library member identified in the library map element for the recipient library member.

13. The computer-implemented method of claim 12, wherein:
the first recipient library is a daughter library derived from at least one of the first and second source libraries in a daughtering operation.

14. The computer-implemented method of claim 12, wherein:
within the first family, at least one of the first and second source libraries is related to the first recipient library by at least three degrees of relationship.

15. The computer-implemented method of claim 13, wherein:
within the first family, the first source library, the second source library and the first recipient library are related libraries in a defined workflow comprising N degrees of relationship between an original source library and the farthest related recipient library for the defined workflow, N being at least three, and
at least one of the first and second source libraries is related to the first recipient library by at least n degrees of relationship, where n ranges from 1 to N.

16. The computer-implemented method of claim 15, wherein:
N is at least five.

17. The computer-implemented method of claim 12, further comprising:
receiving data specifying a second recipient library, the second recipient library having members being derived from materials in two or more source libraries in a second family of library family structure defined by the relationships of the three or more related libraries in the second family, the second library family structure being different than the first library family structure;
defining a plurality of elements of a second library map, the plurality of elements including a library map element identifying each member of the second recipient library, each library map element also identifying a member of the source library from which a material was transferred to the corresponding recipient library member in one or more daughtering operations; and
generating a second experiment object according to the data model representing an experiment on the second recipient library, the second experiment object having a plurality of associated elements representing members of the second recipient library, the generating including assigning to each experiment element of the second experiment object an element identifier based on the source library member identified in the library map element for the recipient library member.

18. The computer-implemented method of claim 12, further comprising:
associating one or more experimental data values with one or more elements of the first experiment object, each experimental data value representing an observation associated with the corresponding member of the first recipient library.

19. A computer program product, tangibly embodied in an information carrier, for managing data associated with members of related libraries of materials including a recipient library, a first source library, and a second source library, the members of the recipient library comprising one or more materials derived from one or more members of the first source library and one or more materials derived from one or more members of the second source library, the computer program comprising instructions to:
define an experiment object representing an experiment performed on members of the recipient library of materials, the experiment object having a plurality of associated elements, each of the plurality of elements representing one or more members of the recipient library; and
store at least one source identifier in association with each of the plurality of elements, the source identifier associated with a given element identifying a source from which the material of the corresponding recipient library member was derived, a first source identifier identifying a member in the first source library and a second source identifier identifying a member in the second source library.

20. The computer program product of claim 19, wherein:
the recipient library is a daughter library derived from at least one of the first and second source libraries in a daughtering operation.

21. The computer program product of claim 19, wherein:
at least one of the first and second source libraries is related to the recipient library by at least two degrees of relationship.

22. The computer program product of claim 19, wherein:
at least one of the first and second source libraries is related to the recipient library by at least three degrees of relationship.

23. The computer program product of claim 19, wherein:
the first source library, the second source library and the recipient library are related libraries in a defined workflow having N degrees of relationship between an original source library and the most distantly related recipient library for the defined workflow, N being at least three.

24. The computer program product of claim 23, wherein:
N is at least five.

25. The computer program product of claim 19, wherein:
storing a source identifier in association with an element includes, for an element representing one of the one or more members, determining the member in the first or second source library from which the material of the member of the recipient library corresponding to the element was derived by:
querying a library map object based on a recipient library identifier and a recipient library element identifier identifying the element in the recipient library, and
receiving a source library identifier and a source library element identifier for the element in response to the query.

26. The computer program product of claim 25, wherein:
the recipient library element identifier identifies a position of the corresponding member in the recipient library and the source library element identifier identifies a position in the source library from which the material of the corresponding member was derived.

27. The computer program product of claim 25, wherein:
the library map object includes a plurality of library map elements, each library map element mapping from an element of the recipient library to an element of a source library from which the material of the corresponding recipient library member was derived.

28. The computer program product of claim 19, further comprising:
receiving a request for experimental data associated with an element of the first or second source library;
querying a database of experiments based on the source library identifier of the source library and the source library element identifier of the element; and
retrieving one or more data values corresponding to recipient library elements satisfying the query.
